(19) United States
(12) Patent Application Publication
(10) Pub. No.: US 2010/0298385 A1
(43) Pub. Date: Nov. 25, 2010

(54) PROTEIN KINASE INHIBITORS USEFUL FOR TREATMENT OF CANCERS

(76) Inventors: Xiaomin Du, Jinan (CN); Yan Hao, Xintai (CN); Lanying Zhang, Jinan (CN)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 12/747,856

(22) PCT Filed: Dec. 11, 2008
(86) PCT No.: PCT/CN08/01994

§ 371 Date: Aug. 9, 2010

(30) Foreign Application Priority Data
Dec. 11, 2007 (CN) 200710115313.1

(51) Int. Cl. A61K 31/4439; C07D 417/12; C07D 213/78; A61K 31/44; A61P 35/04

(52) U.S. Cl. 514/342; 546/270.7; 546/298; 514/354

(57) ABSTRACT

This invention relates to protein kinase inhibitors useful for treating cancers. The present protein kinase inhibitors are those having the structures of the following formula or pharmaceutically acceptable salts thereof.

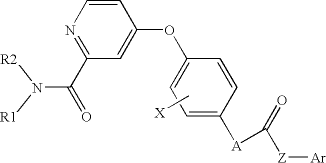

The present compounds can be used to treat protein kinase related diseases such as cancers.

### FIELD OF THE INVENTION

The present invention relates to a new group of compounds as inhibitors of protein kinases, especially Raf Kinase. The present invention also relates to pharmaceutical compositions comprising such compounds, preparation of such compounds, and use of such compounds for the treatment of diseases related to protein kinases especially Raf kinase, including cancers.

### BACKGROUND OF THE INVENTION

A protein kinase is a kinase enzyme that modifies other proteins by chemically adding phosphate groups to them (phosphorylation). Phosphorylation usually results in a functional change of the target protein by changing enzyme activity, cellular location, or association with other proteins. Up to 30% of all proteins may be modified by kinase activity, and kinases are known to regulate the majority of cellular pathways, especially those involved in signal transduction, the transmission of signals within the cell. The human genome contains about 500 protein kinase genes; and they constitute about 2% of all eukaryotic genes.

The chemical activity of a kinase involves removing a phosphate group from ATP and covalently attaching it to one of three amino acids that have a free hydroxyl group. Most kinases act on both serine and threonine, others act on tyrosine, and a number (dual specificity kinases) act on all three. Because protein kinases have profound effects on a cell, their activity is highly regulated. Kinases are turned on or off by phosphorylation (sometimes by the kinase itself—cis-phosphorylation/-autophosphorylation), by binding of activator proteins or inhibitor proteins, or small molecules, or by controlling their location in the cell relative to their substrates. (Blume-Jensen and Hunter, Nature, 411: 355-365, (2001). Kinase has been the targets for drug development, several kinase inhibitors have been approved as drugs. (see review, Fischer, Curr. Med. Chem., 11: 1563 (2004); dancey and Sausville, Nature Rev. Drug Disc. 2: 296 (2003).

Intracellular signaling pathways activated in response to growth factor/cytokine stimulation are known to control functions such as proliferation, differentiation and cell death (Chiloeches and Marais, In Targets for Cancer Therapy; Transcription Factors and Other Nuclear Proteins, 179-206 (La Thangue and Bandara, eds., Totowa, Humana Press 2002). One example is the Ras-Raf-MEK-ERK pathway which is controlled by receptor tyrosine kinase activation. Activation of Ras proteins at the cell membrane leads to phosphorylation and recruitment of accessory factors and Raf which is then activated by phosphorylation. Activation of Raf leads to downstream activation of MEK and ERK. ERK has several cytoplasmic and nuclear substrates, including ELK and Ets-family transcription factor, which regulates genes involved in cell growth, survival and migration (Marais et al., J. Biol. Chem., 272:4378-4383 (1997); Peyssonnaux and Eychene, Biol. Cell, 93-53-62 (2001)). As a result, this pathway is an important mediator of tumor cell proliferation and angiogenesis. For instance, overexpression of constitutively active B-Raf can induce an oncogenic event in untransformed cells (Wellbrock et al., Cancer Res., 64: 2338-2342 (2004)). Aberrant activation of the pathway, such as by activating Ras and/or Raf mutations, is known to be associated with a malignant phenotype in a variety of tumor types (Bos, Hematol. Pathol., 2: 55-63 (1988); Downward, Nature Rev. Cancer, 3: 11-22 (2003); Karasarides et al., Oncogene, 23: 6292-6298 (2004).

There are three Raf isoforms, A-Raf, B-Raf and C-Raf (Raf-1), all of which can act as downstream effectors of Ras. Although they show significant sequence similarities, they also exhibit distinct roles in development, in addition to significant biochemical and functional differences. In particular, the high basal kinase activity of B-Raf may explain why mutated forms of only this isoform have been found in human cancers. B-RAF belongs to the RAF family of serine/threonine kinases. B-RAF is part of a conserved signal transduction pathway that regulates cellular responses to extracellular signals. {Wellbrock et al, Mol Cell Biol. 5:875-885 (2004)}. B-RAF is normally activated downstream of receptors in the cell membrane and is involved in phosphorylating and activating the protein kinase MEK, which subsequently activates the protein kinase ERK. {Niculescu-Duvas er cr/., J. Med. Chem. 49:407-416 (2006)}. ERK phosphorylates transcription factors such as ELK-I, regulating gene expression and controlling how cells respond to extracellular signals. Since B-RAF activation is comparatively easier, it is the strongest activator of downstream MEK and also is a preferred target for mutational activation in human cancers (Biochim Biophys Acta 2003; 1653:25-40). B-RAF is mutated in approximately 7% of human cancers, such as melanoma (50-70%), ovarian (about 35%), thyroid (about 30%) and colorectal (about 10%) cancers. {Davies et al, Cancer Cell 2:95-98 (2003)}. The most common mutation (about 90%) is a glutamic acid for valine substitution at position 600 (V600E). {Niculescu-Duvas et al., J. Med. Chem. 49:407-416 (2006)}. The kinase activity of v600EB-RAF is elevated about 500-fold, providing cancer cells with both proliferation and survival signals and allowing them to grow as tumors in model systems. {Garnett et al, Cancer Cell 4:313-319 (2004)}. Indeed, activation of B-RAF has emerged as the most prevalent oncogenic mutation in thyroid cancer. {Salvatore et al, Clin. Can. Res. 12 (S):1623-1629 (2006)}. Thus, B-RAF is an important factor in both tumor induction and maintenance and presents a new therapeutic target for human cancers. Thus, there is a need in the art for effective inhibitors of B-RAF for use as anticancer and antitumor agents.

Drugs targeting the ERK pathway at the level of Raf may be particularly useful because Raf is the key activator of the ERK pathway, whereas other upstream targets such as growth factor ligands, receptor tyrosine kinases or even Ras, have many other potential effectors. In addition, constitutively active forms of Raf exhibit transforming activity comparable to Ras and are themselves sufficient to transform some cells.

Interestingly, mutations of B-Raf and K-Ras are often found in the tumor types, but in a mutually exclusive fashion, suggesting that B-Raf and K-Ras may provide an equivalent or at least a redundant oncogenic stimulus in cancer pathogenesis. (Cancer Res 2004; 64:1932-7.

Nevertheless, the isoforms show redundant functions in facilitating oncogenic Ras-induced activation of the MEK-ERK signaling cascade (Wellbrock, Cancer Res, 64:2338-2342 (2004)). In addition to Raf signaling via the MEK-ERK pathway there is some evidence that C-Raf (and possibly B-Raf and A-Raf) may signal via alternative pathways directly involved in cell survival by interaction with the BH3 family of anti-apoptotic proteins (Wellbrock et al., Nature Rev.: Mol. Cell. Biol. 5:875 (2004)).

Inhibitors of the Raf kinases may be expected to interrupt the Ras-Raf signaling cascade and thereby provide new methods for the treatment of proliferative disorders, such as cancer. There is thus a need for developing new compounds inhibiting Raf kinase activity.

### SUMMARY OF THE INVENTION

The objects of present invention are to provide a new group of compounds which are protein kinase especially Raf kinase inhibitors, pharmaceutical compositions comprising such compounds, synthesis of such compounds, and use of such compounds for the treatment of diseases related to protein kinases, especially Raf kinase, including cancers.

In one aspect, the present invention provides compounds that have structures as follows,

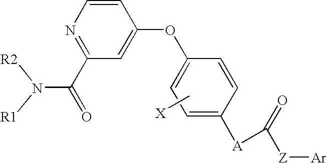

wherein,
R1 and R2 are independently H, a C₁-C₆ alkyl group, a C₂-C₆ alkenyl, a C₃-C₈ cycloalkyl group, wherein the alkyl group, alkenyl group and cycloalkyl group can be substituted with amino, nitro or halo;
X is halogen or a C₁-C₆ alkoxyl group;
A and Z are independently NH or CH₂;
Ar is a five or six membered ring, which can have 1 or 2 heteroatoms selected from oxygen, nitrogen and sulfur and can be substituted with one or more group selected from a C₁-C₆ alkyl groups, halo and halo C₁-C₆ alkyl group,
or pharmaceutically acceptable salts thereof.

In one embodiment, the present invention provides compounds that have structures as follows,

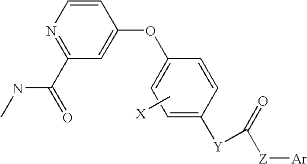

wherein,

### X is F, Cl or OMe;

### Y is CH₂ or NH;

### Z is CH₂ or NH;

Ar is a five or six member ring monosubstituted or disubstituted.

In another embodiment, the present invention provides compounds that have structures as follows,

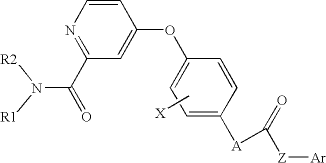

wherein,
R1 and R2 are independently H, or a C₁-C₆ alkyl group;

### X is F, Cl or OMe;

A and Z are independently NH or CH₂;
Ar is a five or six member ring, which ring can have 1 or 2 heteroatoms selected from oxygen, nitrogen and sulfur and can be substituted with one or more groups selected from a C₁-C₆ alkyl group, halo group and halo C₁-C₆ alkyl group,
or pharmaceutically acceptable salts thereof.

In further another embodiment, the present invention provides compounds that have structures as follows,

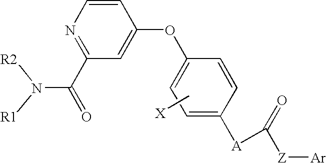

wherein,
R1 and R2 are independently H, or C₁-C₆ alkyl group;

### X is F, Cl or OMe;

A and Z are independently NH or CH₂;
Ar is a five or six member ring which ring can have 1 or 2 heteroatoms selected from oxygen, nitrogen and sulfur and can be substituted with one or two groups selected from a C₁-C₆ alkyl group, halo group and halo C₁-C₆ alkyl group,
or pharmaceutically acceptable salts thereof.

In one particular embodiment, the present invention provides compounds that have structures as follows:

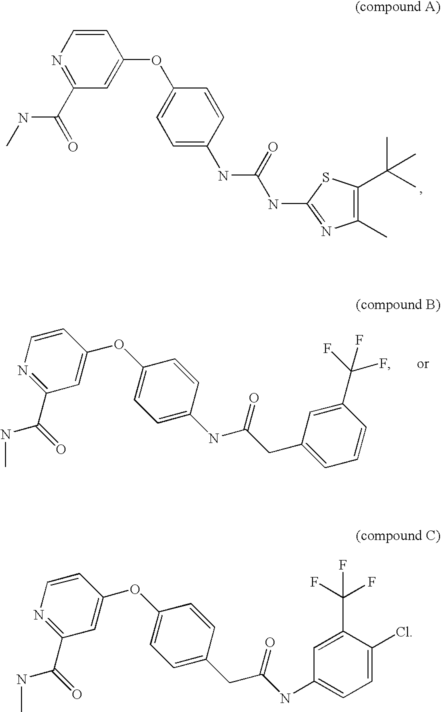

In another aspect, this invention relates to a pharmaceutical composition comprising the compounds of the present invention.

In further another aspect, this invention relates to the use of a compound or pharmaceutical composition of the present invention for the manufacture of a medicament for treating diseases related to a protein kinase such as cancers.

In further another aspect, this invention relates to the use of a compound or pharmaceutical composition of the present invention for the manufacture of a medicament for treating diseases related to a protein kinase such as cancers, or a method for treating diseases related to a protein kinase such as cancers using a compound of the present invention.

The term “C₁-C₆ alkyl”, as used herein, refers to a straight or branched, monovalent, saturated hydrocarbon group which includes 1 to 6 carbon atoms, such as methyl, ethyl, n-propyl, isopropyl, n-butyl, sec-butyl, iso-butyl, tert-butyl, n-pentyl and n-hexanyl.

The term “C₁-C₆ alkoxyl”, as used herein, refers to a group C₁-C₆ alkyl-O—, in which the C₁-C₆ alkyl is defined as above. Typical examples of C₁-C₆ alkoxyl are methoxyl, ethoxyl, n-propoxyl, iso-propoxyl, n-butoxyl, sec-butoxyl, iso-butoxyl, and tert-butoxyl.

The term “C₂-C₆ alkenyl”, as used herein, refers to a straight or branched, monovalent, unsaturated hydrocarbon group, which includes 2 to 6 carbon atoms, and has at least one, normally 1, 2, or 3 carbon-carbon double bonds. Typical examples of C₂-C₆ alkenyl are ethenyl, n-propenyl, iso-propenyl, n-but-2-enyl, and n-hex-2-enyl.

The term “C₃-C₈ cycloalkyl”, as used herein, refers to a monovalent, saturated, carbocyclic hydrocarbon group, which includes 3 to 8 carbon atoms. Typical examples of C₃-C₈ cyloalkyl are cyclopropyl, cyclobutyl, cyclopentyl and cyclohexyl.

The present invention is also directed to pharmaceutically acceptable salts of the compounds as recited above. Suitable pharmaceutically acceptable salts are well known to those skilled in the arts and include basic salts of inorganic and organic salts, such as hydrochloric acid, hydrobromic acid, sulfuric acid, phosphoric acid, methanesulphonic acid, trifluoromethanesulfonic acid, benzenesulfonic acid, p-toluenesulfnic acid, 1-naphthalenesulfonic acid, 2-naphthalenesulfonic acid, acetic acid, lactic acid, trifluoroacetic acid, malic acid, tartaric acid, citric acid, oxalic acid, fumaric acid, succinic acid, maleic acid, salicylic acid, benzoic acid, phenylacetic acid, mandelic acid, etc. In addition, pharmaceutically acceptable salts include acid salts of the present compounds with inorganic bases, such as salts with alkaline metal cations, alkaline earth metal cations, and ammonium cation, as well as acid salts with organic bases, including aliphatic and aromatic substituted ammonium, and quaternary ammonium cations.

The compounds may be prepared from the commercially available chemical starting materials and intermediates by a process shown in the following typical scheme. Examples will be given herein in the following section of Example to illustrate the specific methods for preparing the present compounds.

### A Representative Scheme for Preparing the Present Compounds

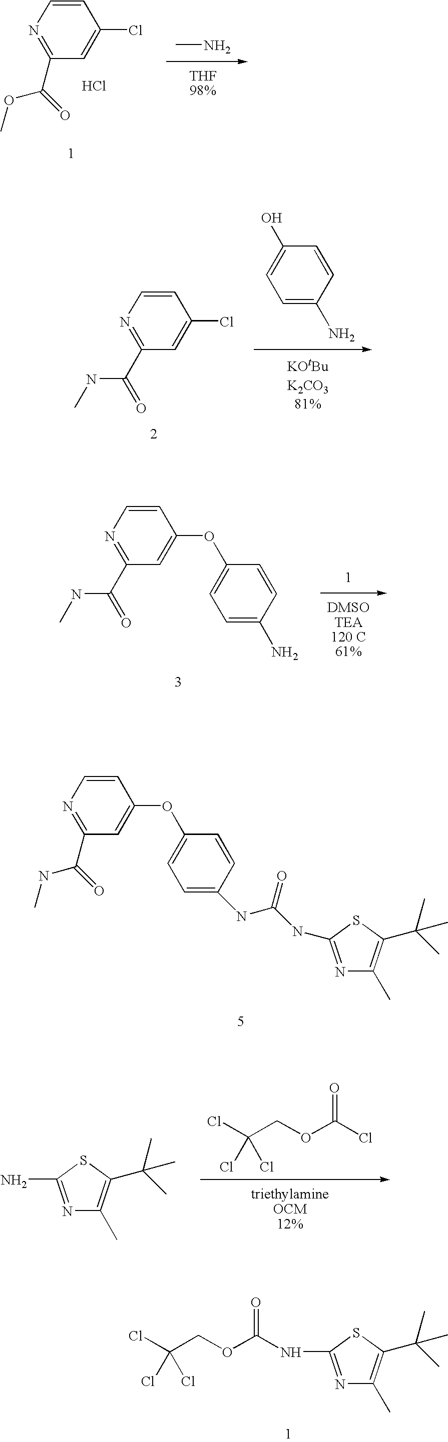

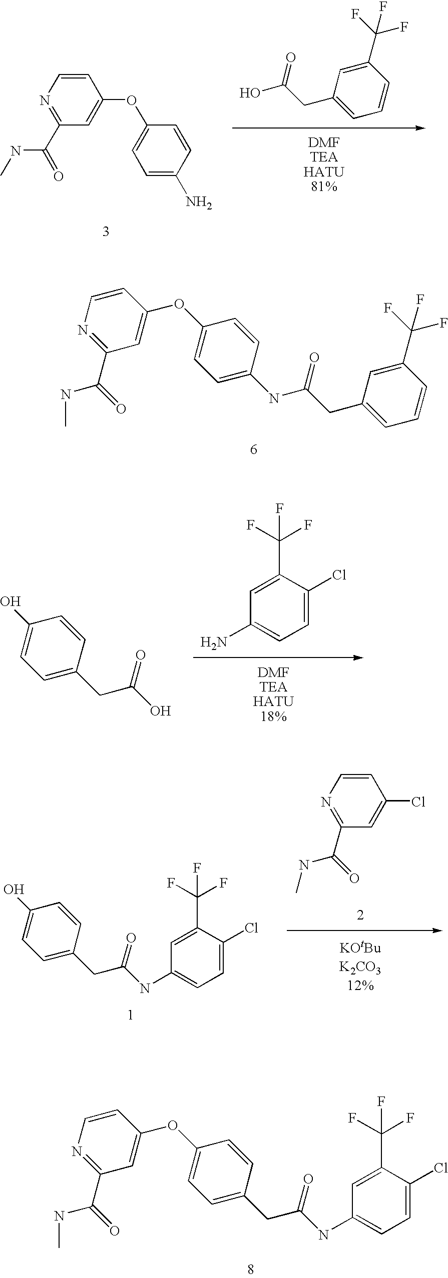

The compounds may be administered orally, topically, parenterally, by inhalation or spray or rectally in dosage unit formulations. The term “administration by injection” includes intravenous, intramuscular, subcutaneous and parenteral injections, as well as use of infusion technology.

The invention also includes pharmaceutical compositions intended for oral use. This can be prepared according to any suitable method known to the art for the manufacture of pharmaceutical compositions. Such compounds may contain one or more agents selected from the group consisting of diluents, sweetening agents, flavoring agents, coloring agents and preserving agents. Tablets contain the active ingredient with non toxic pharmaceutically acceptable excipients which are suitable for the manufacture of tablets. These exipients may be, for example, inert diluents, such as calcium carbonate, sodium carbonate, lactose, calcium phosphate or sodium phosphate; granulating and disintegrating agents, for example, corn starch, or alginic acid; and binding agents, such as magnesium stearate, stearic acid or talc. The tablets may be uncoated or they may be coated by known techniques to delay disintegration and absorption in the gastrointestinal tract and thereby provide a sustained action over a long period of time. For example, a time delay material such as glyceryl monostearate or glyceryl distearate may be employed. These compounds may be prepared in solid, rapidly released form.

The present compounds can be formulated in different dosage forms, such as hard gelatin capsule, aqueous suspension, dispersible powder, granules, non-aqueous liquid form and oil-in-water emulsion.

It has to be noted that the specific dose level for any particular patient will depend on a variety factors, including the activity of the specific compound employed, the age, body weight, general health, sex, diet, time of administration, route of administration, and rate of excretion, drug combination and the severity of the condition undergoing therapy.

The present invention provides compounds which are effective kinases especially Raf kinase inhibitors. Those compounds inhibit kinase in vitro and in vivo, and they are effective for use in the treatment of a cell proliferation.

The present invention provides compounds which are kinases especially Raf kinase inhibitors. The instant inhibitors have significant medical values in the treatment of tumuors and other diseases caused by activiation of a kinase (suck as raf, tyrosine kinase etc) pathway in a human or animal. Accordingly, the compounds of the invention are useful in treating solid cancers such as lung cancer, pancreas cancer, bladder cancer, colon cancer, and leukemia.

The present invention provides compounds which have the following advantageous effects comparing with the known inhibitors of Raf kinase having the similar structures:

1. Extremely low toxicity (the maximum dose of administration is >5 g/kg in mice), very safe, and good tolerance are observed as compared with the known compounds;
2. A broad-spectrum and high strength of anti-cancer activities (in μM/L); and
3. Pharmacokinetic properties are more advantageous for exerting the therapeutical effects due to presence of the amide structure in the present compounds (solubility in THF/>1 g/mL) instead of the urea structure in the known compounds (solubility in THF<<1 g/mL).

### CONCRETE MODES FOR CARRYING OUT THE INVENTION

All reactions were performed in flame-dry or oven-dry glassware under a positive pressure of dry nitrogen, and were stirred magnetically unless otherwise indicated. Sensitive liquids and solutions were transferred via syringe or cannula, and introduced into reaction vessels through rubber septa.

All temperatures were reported uncorrected in degrees Celsius. Unless otherwise indicated, all parts and percentages are by weight.

Commercial grade reagents and solvents were used without further purification. Thin-layer chromatography (TLC) was performed using Whatman pre-coated glass-backed silica gel 60A GF254 250 uM plates. Visualization of plates was effected by one or more of the following techniques: 1) ultraviolet illumination, 2) exposure to iodine vapor, 3) immersion of the plate in a 10% solution of phosphomolybic acid in ethanol followed by heating, 4) immersion of the plate in a cerium sulfate solution followed by heating. Column chromatography was performed by using 230-400 mesh EM Science silica gel G.

Melting points (mp) were determined using Thomas-Hoover melting point apparatus. Proton (¹H) nuclear magnetic resonance (NMR) spectra were measured with a Varian 400 (400 Hz) spectrometer with either Me4Si (δ0.00 ppm) or the residual protonated solvent (CDCl₃, δ7.26 ppm, MeOH δ3.30 ppm, DMSO δ2.49 ppm) as a standard. Carbon (¹³C) NMR spectra were measured with a Varian 400 (400 Hz) spectrometer with solvent (CDCl₃ δ 77.0, MeOD δ49.0, DMSO δ39.5) as a standard. Low resolution mass spectra (MS) and high resolution mass spectra (HRMS) were either obtained as electron impact (EI) mass spectra or as fast atom bombardment (FAB) mass spectra.

The structures of all the compounds were confirmed by NMR spectra, and MS.

### Example 1

### Synthesis of 4-{4-[3-(5-tert-Butyl-4-methyl-thiazol-2-yl)-ureido]-phenoxy}-pyridine-2-carboxylic acid methylamide)

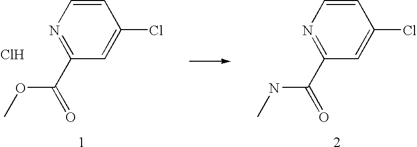

4-chloro-pyridine-2-carboxylic acid methyl ester HCl salt (7.00 g, 32.95 mmol) was added portionwise to 2.0 M methylamine in THF (100 mL) and methanol (20 mL) at 0° C. under nitrogen. The mixture was stirred at 3° C. for 4 hrs, concentrated to near dryness, and dissolved in ethyl acetate (100 mL). The resulting white solid was filtered off. The organic layers were washed with brine (2×100 mL), dried over sodium sulfate, and concentrated to give compound 2, 4-chloro-pyridine-2-carboxylic acid methylamide as a clear slightly yellow liquid.

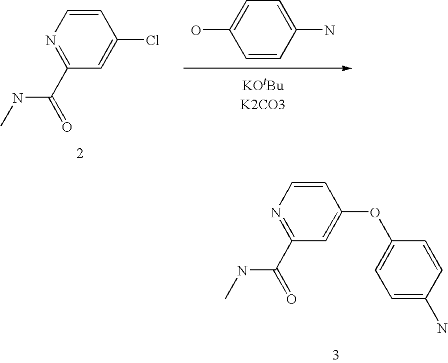

A solution of 4-aminophenol (9.60 g, 87.98 mmol) in dry DMF (150 mL) was treated with potassium tert-butoxide (10.29 g, 91.69 mmol), and the reddish-brown mixture was stirred at room temperature for 2 h. The contents were treated with 4-chloro-pyridine-2-carboxylic acid methylamide (15.00 g, 87.92 mmol) and potassium carbonate (6.50 g, 47.03 mmol) and then heated to 80° C. under nitrogen for 6 h. The mixture was cooled to room temperature and poured into EtOAc (500 mL) and brine (500 mL) with stirring. The layers were separated, and the aqueous phase was extracted with EtOAC (2×150 mL). The combined organic layers were washed with brine (4×1000 mL), dried over sodium sulfate, filtered and concentrated to afford compound 3, 4-(4-amino-phenoxy)-pyridine-2-carboxylic acid methylamide (18.62 g, 76.54 mmol, 87%) as a light brown solid.

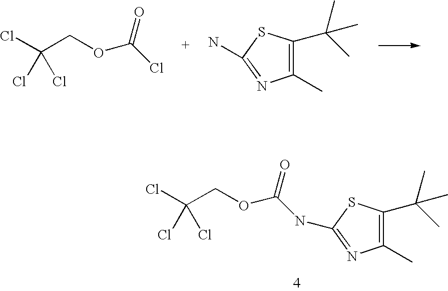

To the solution of 5-tert-Butyl-4-methyl-thiazol-2-ylamine (1.12 g, 6.6 mmol) in dry

DMF (20 mL) was added triethylamine (0.92 mL, 1 eq). 2,2,2-trichloroethyl chloroformate (0.9 mL, 6.6 mmol) was added in a dropwise manner at room temperature. The reaction mixture was stirred at room temperature for 3 hrs. Dry DMF (40 mL) was added, washed with brine (3×20 mL), water (2×20 mL), dried, filtered, concentrated to afford compound 4, (5-tert-butyl-4-methyl-thiazol-2-yl)-carbamic acid 2,2,2-trichloro-ethyl ester (1.40 g, 4.0 mmol, 61%) as a white solid.

MS: 347.0 (M+1)

¹HNMR (DMSO-d6) ppm: 1.26 (9H, s), 2.12 (3H, s), 4.90 (2H, s)

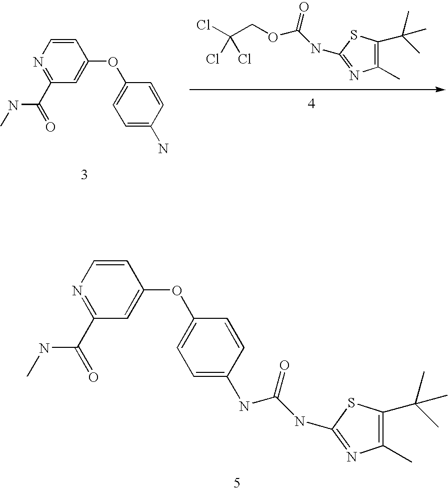

To the solution of 4-(4-amino-phenoxy)-pyridine-2-carboxylic acid methylamide (0.18 g, 0.74 mmol, 1 eq.) and (5-tert-butyl-4-methyl-thiazol-2-yl)-carbamic acid 2,2,2-trichloro-ethyl ester (0.26 g, 1 eq.) in DMSO (2 mL) was added triethylamine (0.10 mL, 1 eq). The reaction mixture was stirred at 100° C. in microwave for 20 min. The solution was poured into ice water (20 mL), and filtered off the solid. The solid was washed with brine (2×20 mL) and water (2×20 mL), dried over sodium sulfate, filtered, and concentrated. The residue was purified by flash silica column (1-4% MeOH/DMF) to afford compound 5, the compound A as off-white solid (0.2 g, 0.46 mmol, 62%).

MS: 440.2 (M+1)

¹HNMR (DMSO-d6) ppm: 1.26 (9H, s), 2.12 (3H, s), 2.74 (3H, d), 7.10 (2H, d), 7.16 (1H, d), 7.26 (1H, d), 7.48 (2H, d), 8.42 (1H, d), 8.70 (1H, d), 9.02 (1H, s), 10.08 (1H, b).

### Example 2

### Synthesis of 4-{4-[2-(4-chloro-3-trifluoromethyl-phenyl)-acetylamino]-phenoxy}-pyridine-2-carboxylic acid methylamide

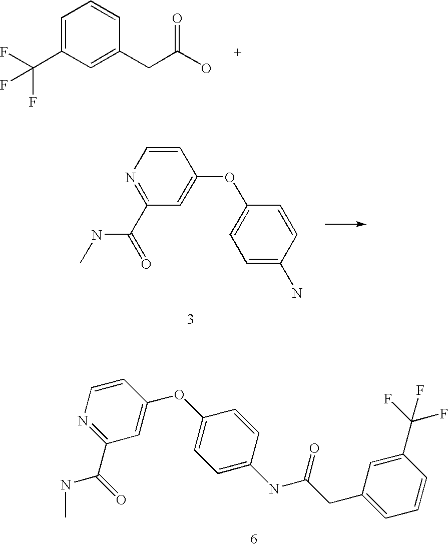

to the solution of 4-(4-amino-phenoxy)-pyridine-2-carboxylic acid methylamide (0.36 g, 1.48 mmol, 1 eq.) and 3-trifluoromethyl-phenyl-acetic acid (0.31 g, 1.48 mmol, 1 eq.) in DMF (2 mL) was added triethylamine (0.21 mL, 1.5 mmol, 1 eq.). HATU (1.56 g, 1.48 mmol, 1 eq.) was added finally. The reaction mixture was stirred at room temperature for 3 hrs. The reaction mixture was poured into ice water (30 mL). The solid was filtered off, dissolved in DMF (60 mL), washed with brine (2×30 mL), water (2×30 mL), dried over sodium sulfate, and concentrated under a reduced pressure after filtering off the drying reagent. The residue was purified by flash silica gel column (1-4% MeOH/DCM) to afford compound 6, the compound B-chloro as a white solid (0.52 g, 1.2 mmol, 81%).

¹HNMR (DMSO-d6) ppm: 2.74 (3H, d), 3.78 (2H, s), 7.04 (1H, m), 7.18 (2H, d), 7.30 (1H, d), 7.50 (3H, m), 7.64 (3H, m), 8.41 (1H, d), 8.71 (1H, b), 10.38 (1H, s);

MS: 430.0 (M−1).

### Example 3

### Synthesis of 4-(4-[(4-chloro-3-trifluoromethylphenylaminocarbonyl)-methyl]phenoxy)pyridin-2-carboxylic acid methylamide

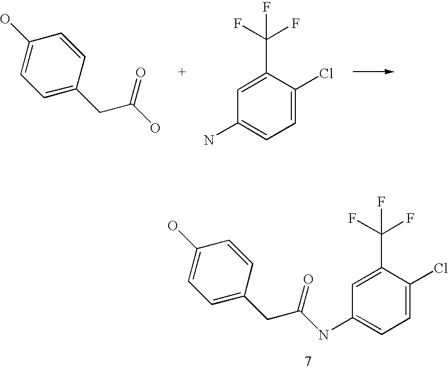

Diisopropyl ethyl amine (2 eq.) was added to the solution of 4-hydroxyphenylacetic acid (0.56 g, 1 eq.) and 4-chloro-3-trifluorophenylamine (1.08 g, 1.5 eq.) in DMF (3 mL). HATU was added finally (1.4 g, 1.1 eq.). The reaction mixture was heated to 60° C. for 4 hours. Ethyl acetate (120 mL) was added, washed with brine (3×30 mL), water (3×30 mL), dried over Na₂SO₄, filtered, and concentrated. The residue was purified by flash silica column (10-30 EtOAc/DCM). Compound 7, N-(4-chloro-3-trifluoromethyl-phenyl)-2-(4-hydroxy-phenyl)-acetamide was afforded as white solid.

MS: 328.0, 329.0 (M−1); 329.0, 330.0 (M+1);

¹HNMR (DMSO-d6) ppm: 3.43 (2H, s), 6.62 (2H, d), 7.04 (2H, d), 7.58 (1H, d), 7.78 (1H, dd), 8.07 (1H, s), 9.12 (1H, s), 10.44 (1H, s).

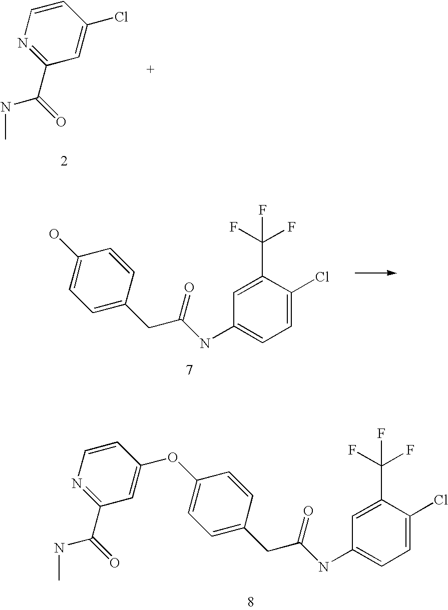

A solution of N-(4-chloro-3-trifluoromethyl-phenyl)-2-(4-hydroxy-phenyl)-acetamide (1 eq.) in dry DMF (150 mL) was treated with potassium tert-butoxide (1.2 eq.), and the reddish-brown mixture was stirred at room temperature for 2 h. The contents were treated with 4-chloro-pyridine-2-carboxylic acid methylamide (15.00 g, 87.92 mmol) and potassium carbonate (0.6 eq.) and then heated to 80° C. under nitrogen for 6 h. The mixture was cooled to room temperature and poured into EtOAc (500 mL) and brine (500 mL). The layers were separated, and the aqueous phase was extracted with EtOAC (2×150 mL). The combined organic layers were washed with brine (4×1000 mL), dried over sodium sulfate, filtered and concentrated. The residue was purified by flash silica gel column to afford compound 8, the compound C as a light yellow to off-white solid.

MS: 464.0 (M+1)

¹HNMR (DMSO-d6, ppm): 10.71 (s, 1H), 8.81 (d, 1H), 8.52 (d, 1H), 8.25 (d, 1H), 7.89 (t, 1H), 7.67 (d, 1H), 7.49 (d, 2H), 7.42 (d, 1H), 7.22 (d, 2H), 7.18 (s, 1H), 4.04 (d, 2H), 2.80 (d, 3H).

### Example 4

### Assays for Determining the Biological Activity of the Present Compounds

### I. Assays for Suppressing Tumors In Vivo

### 1. Experimental Materials:

Tested compound samples: compound A, compound B, and compound C;
Positive control compound: Paclitaxel, cyclophosphamide (CYC);
1 RPMI 1640 medium, FBS, MTT, PBS (pH 7.3), Glucose, benzylpenicillin, streptomycin, DMSO, cell culture plate (96 cells).
2. Cell Lines for Experiments (Cancer Cells Strains were from: National Natural and BioDrug Lab in Peking University, and Cancer Lab in Shenyang Pharmaceutical University).

MCF-7, HepG-2, A549, MCF-7, Hela, HL-60, K562, U937, L929, A375s-2, KB, A431, BGC-823, Bel-7402, KB.

### 3. Experimental Methods

Cell incubation: the cancer cells strains are incubated in RPMI 1640 with 10% FBS, grow along the wall at 37° C. under 5% CO₂ incubator.

Sample solutions: First prepare material at 100 mM in DMSO, then diluted to DMSO 4.95% using 3% DMSO FBS solution.

MTT test: cancer cells are incubated at a density of 5×10³ cells/well in 96 well cell Culture plate for 24 hours, then added different sample solutions for 72 hours. At the end of incubation, 5 mg/mL MTT 15 μL was added to each well and stayed for 4 hours, vacuumed the solution, added DMSO 150 μL, shaked for 10 minutes, tested at 540 nM for the OD value in the spectrophotometer, and calculated the inhibition rate of cell proliferation.

Inhibition rate of cell proliferation=(control OD−sample OD)/control OD)*100%

### 4. Experimental Results

The result showed the inhibition of Paclitaxel, CYC at 0.4, 0.8, 1.6, 3.2, and 6.4 μm/L for the different cells as identified above was dose-dependent. The results of the three compounds for suppressing tumors in vivo were given in the following table

### Experimental Results of Biological Activity of the Compounds

| Cancer Cell Line | Compound A | Compound B | Compound C |
|---|---|---|---|
| MCF-7 | +++ | + | + |
| HepG-2 | ++ | + | ++ |
| A549 | + | + | + |
| Hela | + | ++ | ++ |
| MCF-7 | +++ | + | ++ |
| HL-60 | ++ | ++ | +++ |
| K562 | + | + | + |
| U937 | ++ | + | +++ |
| L929 | + | + | + |
| A375s-2 | + | ++ | ++ |
| KB | ++ | + | + |
| A431 | ++ | + | ++ |
| BGC-823 | ++ | ++ | ++ |
| Bel-7402 | +++ | ++ | +++ |
| KB | ++ | + | ++ |

+++: IC50 ≦10 μmol/L,
++: IC50 11-30 μmol/L,
+: IC50 31-50 μmol/L

### II. Test for Acute Toxicity

The mice were administrated with compounds and observed for 14 days. The acute toxic reaction and death rate were observed after being administrated at an over-great dose. Result: The mice in the compounds group had no abnormal reactions after a short uncomfortable period at the beginning, and no animal died. The experimental results showed that the maximum dose of oral administration is >5-10 g/kg. Clearly, the present compounds have much less toxicity than the commonly used chemotherapy drugs.

1-3. (canceled)

4. A compound selected from the group consisting of 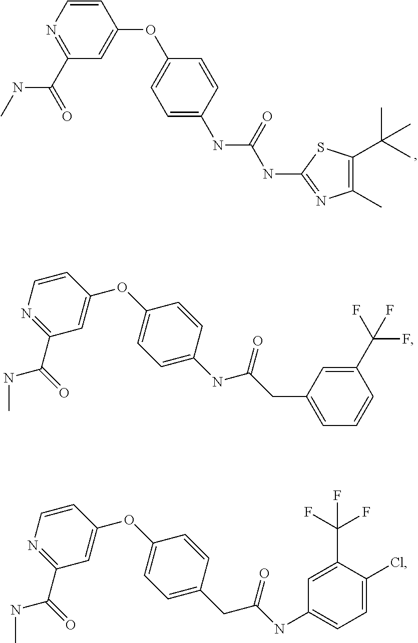
and pharmaceutically acceptable salts thereof.

5. A pharmaceutical composition comprising a compound according to claim 4, and one or more pharmaceutically acceptable excipients.

6. A method for treating a disease related to a protein kinase comprising administering to a subject in need thereof a therapeutically effective amount of a compound according to claim 4.

7. The method according to claim 6, wherein said disease related to a protein kinase is a cancer.

8. The method according to claim 6, wherein the protein kinase is Raf kinase.
